(19) United States
(12) Patent Application Publication
(10) Pub. No.: US 2025/0339650 A1
(43) Pub. Date: Nov. 6, 2025

(54) INTRODUCER SHEATH WITH BLOOD BYPASS FRAME

(71) Applicant: BOSTON SCIENTIFIC SCIMED, INC., Maple Grove, MN (US)

(72) Inventors: Daniel Presteng, Corcoran, MN (US); Lauren Ann Sandy, Minneapolis, MN (US); Jeffrey Schneider, Dayton, MN (US); Zachary Kumor, Plymouth, MI (US); Benjamin Jerome Bidne, Loretto, MN (US)

(73) Assignee: BOSTON SCIENTIFIC SCIMED, INC., Maple Grove, MN (US)

(21) Appl. No.: 19/194,313

(22) Filed: Apr. 30, 2025

Related U.S. Application Data
(60) Provisional application No. 63/641,092, filed on May 1, 2024.

(51) Int. Cl. A61M 25/06; A61M 39/06; A61M 60/174; A61M 60/226; A61M 60/419; A61M 60/865

(52) U.S. Cl. CPC A61M 25/0662; A61M 39/06; A61M 60/174; A61M 60/226; A61M 60/419; A61M 60/865; A61M 2025/0681; A61M 2039/062

(57) ABSTRACT

A hub and sheath assembly configured to increase the flow of fluid through a lumen, such as vessel. The hub and sheath assembly may comprise a valve hub including a lumen extending therethrough, an elongate shaft extending from a proximal end region to a distal end region and including a lumen extending therebetween, the elongate shaft coupled to the valve hub, and an expandable frame extending around an outer surface of the elongate shaft, the expandable frame extending from a proximal end to a distal end. The expandable frame may be configured to move between a radially collapsed configuration and a radially expanded configuration.

### CROSS REFERENCE TO RELATED APPLICATIONS

The present application claims the benefit of U.S. Provisional Patent Application Ser. No. 63/641,092, filed May 1, 2024, the disclosure of which is incorporated herein by reference.

### TECHNICAL FIELD

The present disclosure pertains to sheaths for removing and/or delivering intravascular medical devices. More specifically, the present disclosure relates to a sheath which may be disposed over an in-situ medical device.

### BACKGROUND

In various procedures for delivering intravascular medical devices, a sheath is inserted into a blood vessel of a patient, for example a femoral artery, and one or more medical devices may be advanced through the sheath and into the patient's vasculature. In various instances, the medical devices include catheters or other devices, such as a blood pump. The sheath may remain in place while the medical device is within the body to facilitate removal of the medical device when the procedure is complete. A device that is in use for an extended period of time (e.g., multiple days) may require minimum disruption to blood flow to avoid ischemia. In some cases, a sheath may reduce blood flow while it remains in the body. Thus, there is a need for improved sheaths for introduction and/or removal of medical devices that minimally disrupt blood flow while the medical device is in use.

### BRIEF SUMMARY

This disclosure provides design, material, manufacturing method, and use alternatives for medical devices, including introducer and/or removal sheaths.

In a first example, a hub and sheath assembly may comprise a valve hub including a lumen extending therethrough, an elongate shaft extending from a proximal end region to a distal end region and including a lumen extending therebetween, the elongate shaft coupled to the valve hub, and an expandable frame extending around an outer surface of the elongate shaft, the expandable frame extending from a proximal end to a distal end. The expandable frame may be configured to move between a radially collapsed configuration and a radially expanded configuration.

Alternatively or additionally to any of the examples above, in another example, the proximal end of the expandable frame may be fixedly coupled to the elongate shaft.

Alternatively or additionally to any of the examples above, in another example, the distal end of the expandable frame may be movably disposed relative to the elongate shaft.

Alternatively or additionally to any of the examples above, in another example, the distal end of the expandable frame may be fixedly coupled to the elongate shaft.

Alternatively or additionally to any of the examples above, in another example, the proximal end of the expandable frame may be movably disposed relative to the elongate shaft.

Alternatively or additionally to any of the examples above, in another example, the hub and sheath assembly may further comprise a collar extending around a circumference of the expandable frame.

Alternatively or additionally to any of the examples above, in another example, the collar may be adjacent to the distal end of the expandable frame.

Alternatively or additionally to any of the examples above, in another example, the collar may be adjacent to the proximal end of the expandable frame.

Alternatively or additionally to any of the examples above, in another example, the hub and sheath assembly may further comprise an actuation member extending from a distal end secured to the expandable frame to a proximal end.

Alternatively or additionally to any of the examples above, in another example, the hub and sheath assembly may further comprise an actuation mechanism coupled to the proximal end of the actuation member.

Alternatively or additionally to any of the examples above, in another example, the actuation mechanism may be configured to axially displace to the actuation member to move the expandable frame between the radially collapsed configuration and the radially expanded configuration.

Alternatively or additionally to any of the examples above, in another example, the actuation mechanism may be disposed within the valve hub.

Alternatively or additionally to any of the examples above, in another example, the actuation member may extend along an outer surface of the elongate shaft.

Alternatively or additionally to any of the examples above, in another example, the actuation member may be coupled to the collar.

Alternatively or additionally to any of the examples above, in another example, the expandable frame may comprise two or more interwoven filaments.

In another example, a hub and sheath assembly may comprise a valve hub including a lumen extending therethrough, an elongate shaft extending from a proximal end region to a distal end region and including a lumen extending therebetween, the elongate shaft coupled to the valve hub, and an expandable frame extending around an outer surface of the elongate shaft, the expandable frame extending from a proximal end fixedly secured to the elongate shaft to a distal end movably disposed relative to the elongate shaft. The expandable frame may be configured to be actuated between a radially collapsed configuration and a radially expanded configuration.

Alternatively or additionally to any of the examples above, in another example, the hub and sheath assembly may further comprise an actuation member extending from a distal end secured to the distal end of the expandable frame to a proximal end.

Alternatively or additionally to any of the examples above, in another example, the hub and sheath assembly may further comprise an actuation mechanism coupled to the proximal end of the actuation member.

Alternatively or additionally to any of the examples above, in another example, the actuation mechanism may be configured to axially displace to the actuation member to move the expandable frame between the radially collapsed configuration and the radially expanded configuration.

In another example, a hub and sheath assembly may comprise a valve hub including a lumen extending therethrough, an elongate shaft extending from a proximal end region to a distal end region and including a lumen extending therebetween, the elongate shaft coupled to the valve hub, an expandable frame extending around an outer surface of the elongate shaft, the expandable frame extending from a proximal end fixedly secured to the elongate shaft to a distal end movably disposed relative to the elongate shaft, a collar disposed around the distal end of the expandable frame, an actuation mechanism disposed within the valve hub, and an actuation member extending from a distal end coupled to the collar to a proximal end coupled to the actuation mechanism. The actuation mechanism may be actuatable to move the expandable frame between a radially collapsed configuration and a radially expanded configuration.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a side view of an introducer sheath extending into a blood vessel;

FIG. 2 is a cross-sectional view of a portion of the introducer sheath of FIG. 1 inserted into a blood vessel, and a medical device inserted into the introducer sheath;

FIG. 3A is a side view of an illustrative introducer sheath in a first configuration;

FIG. 3B is a side view of an illustrative introducer sheath in a second configuration;

FIG. 4A is a partial cross-sectional view of the illustrative introducer sheath of FIG. 3A disposed within a vessel; and

FIG. 4B is a partial cross-sectional view of the illustrative introducer sheath of FIG. 3B disposed within a vessel.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

Relative terms such as “proximal”, “distal”, “advance”, “retract”, variants thereof, and the like, may be generally be considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user.

The following detailed description should be read with reference to the drawings. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Some medical devices may extend percutaneously into the body and remain in position for an extended period of time (e.g., hours, days, weeks, etc.). An introducer sheath may be used to facilitate introduction of the medical device into the vasculature. For example, a sheath may be introduced into the femoral artery and one or more medical devices may be advanced through the sheath and into the patient's vasculature. In some embodiments, the medical device may include catheters or other devices, such as, but not limited to, a blood pump. The introducer sheath may remain within the body while the medical device remains in use with a hemostasis valve hub assembly helping to facilitate insertion of the medical device as well as helping to prevent blood from leaking during the medical procedure. When the medical procedure is complete, or the medical device is no longer needed, the introducer sheath may facilitate removal of the medical device. It is desirable for a medical device that is in use for a prolonged period of time (e.g., more than twenty-four hours) to minimally disrupt blood flow to avoid critical limb ischemia. However, an introducer sheath may reduce blood flow for as long as it remains within the body increasing the risk of ischemia. Some embodiments of the present disclosure are directed toward a sheath, such as, but not limited to, an introducer sheath, that can improve blood flow when the sheath remains in the body.

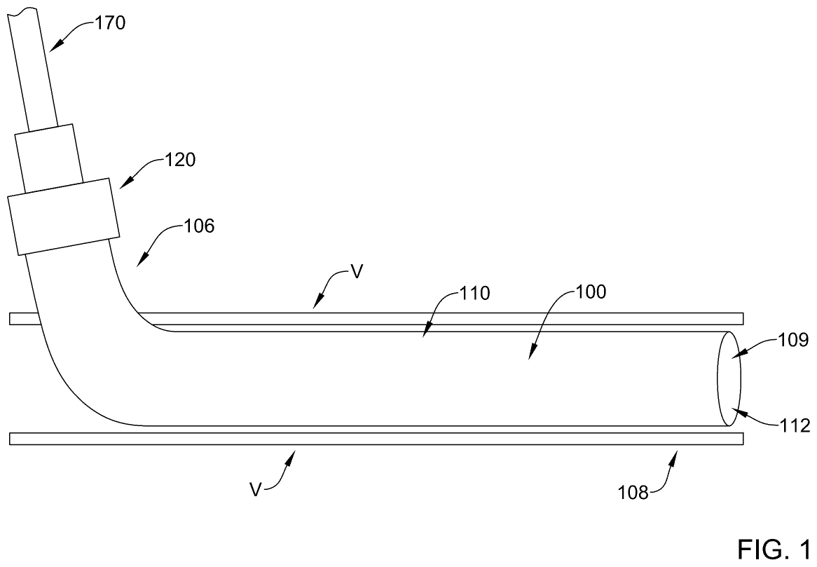

FIG. 1 illustrates a side view of an introducer sheath 100 inserted at least partially into a blood vessel V, shown in cross-section. In some embodiments, the introducer sheath 100 may be used for facilitating the passage of various medical devices, such as a catheter or a blood pump, as will be described further herein, through the introducer sheath 100 and into the blood vessel V. The introducer sheath 100 includes a proximal end region 106 proximate a proximal end of the introducer sheath 100 and a distal end region 108 proximate a distal end of the introducer sheath 100 that is opposite the proximal end region 106. An elongate shaft 110 of the introducer sheath 100 extends between the proximal end region 106 and the distal end region 108, and the elongate shaft 110 defines a lumen 112 of the introducer sheath 100. The introducer sheath 100 includes a proximal opening (not shown) adjacent the proximal end region 106 and a distal opening 109 adjacent the distal end region 108, with the lumen 112 extending from the proximal opening to the distal opening 109. The introducer sheath 100, or components thereof, may be formed by various materials, such as polymeric and/or metallic materials. In some instances, the introducer sheath 100, such as the elongate shaft of the introducer sheath 100, may include an additional surface coating, such as but not limited to, silicone, polyethylene terephthalate (PET), or other applicable polymer.

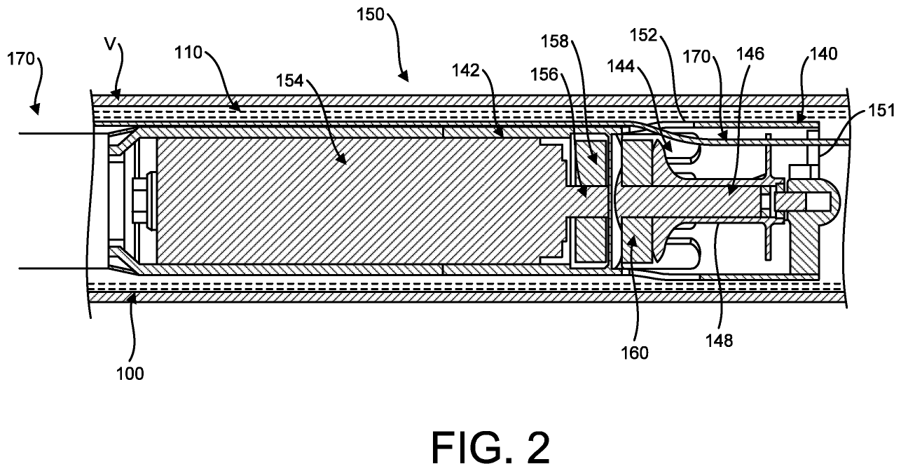

A hemostasis valve hub 120 (hereinafter “hub 120” for brevity) may be provided at the proximal end region 106 to provide access to the lumen 112 of the introducer sheath 100. The hub 120 may be configured for hemostasis by, for example, helping to prevent blood from leaking out of the introducer sheath 100 during use. For example, a medical device 170, such as a catheter or blood pump, may be inserted through the hub 120 and lumen 112 of the introducer sheath 100 and into the blood vessel V, and the hub 120 may maintain hemostasis between the medical device 170, the introducer sheath 100, and the external surroundings. In some embodiments, the medical device 170, may include and/or be coupled to a blood pump 150, shown in FIG. 2.

FIG. 2 illustrates a cross-sectional view of the elongate shaft 110 of the introducer sheath 100 of FIG. 1 upon insertion of a medical device, illustratively a blood pump 150, into the introducer sheath 100. As noted above, the medical device 170 of FIG. 1 may be coupled to or include the blood pump 150, with the medical device 170 extending outside the blood vessel V and the introducer sheath 100. The blood pump 150 may be advanced through the blood vessel V and positioned in a target location, such as a target cardiac location (e.g., the left ventricle), via the introducer sheath 100. The blood pump 150 may generally include an impeller assembly housing 140 and a motor housing 142. In some embodiments, the impeller assembly housing 140 and the motor housing 142 may be integrally or monolithically constructed (e.g., formed as single unitary structure). In other instances, the impeller assembly housing 140 and the motor housing 142 may be separate components. The impeller assembly housing 140 carries an impeller assembly 144 therein. The impeller assembly 144 may include an impeller shaft 146 and an impeller 148 that rotate relative to the impeller assembly housing 140 to drive blood through the blood pump 150. More specifically, the rotation of the impeller 148 causes blood to flow from a blood inlet 151 formed on the impeller assembly housing 140, through the impeller assembly housing 140, and out of a blood outlet 152 formed on the impeller assembly housing 140. In some embodiments, the impeller shaft 146 and the impeller 148 may be integrally formed as a single unitary structure, whereas, in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may be coupled to a distally extending cannula, and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly 144. More specifically, in embodiments incorporating the impeller shaft 146, the impeller shaft 146 and the impeller 148 are configured to rotate with the driven magnet 160. In other embodiments, the motor 154 may be coupled to the impeller assembly 144 via other components. While the introducer sheath 100 is illustrated above with the use of the blood pump 150, various other medical devices may be used in conjunction with the introducer sheath 100 and the hemostasis valve hub 120.

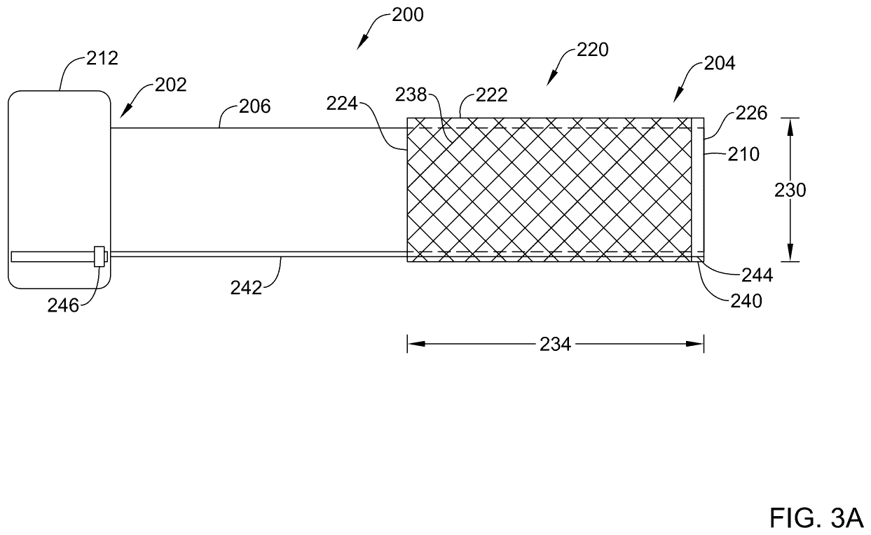

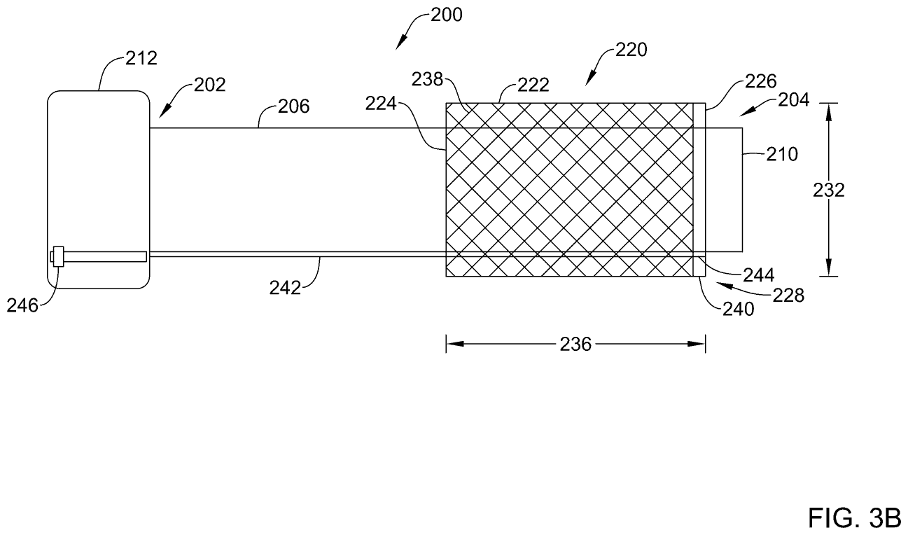

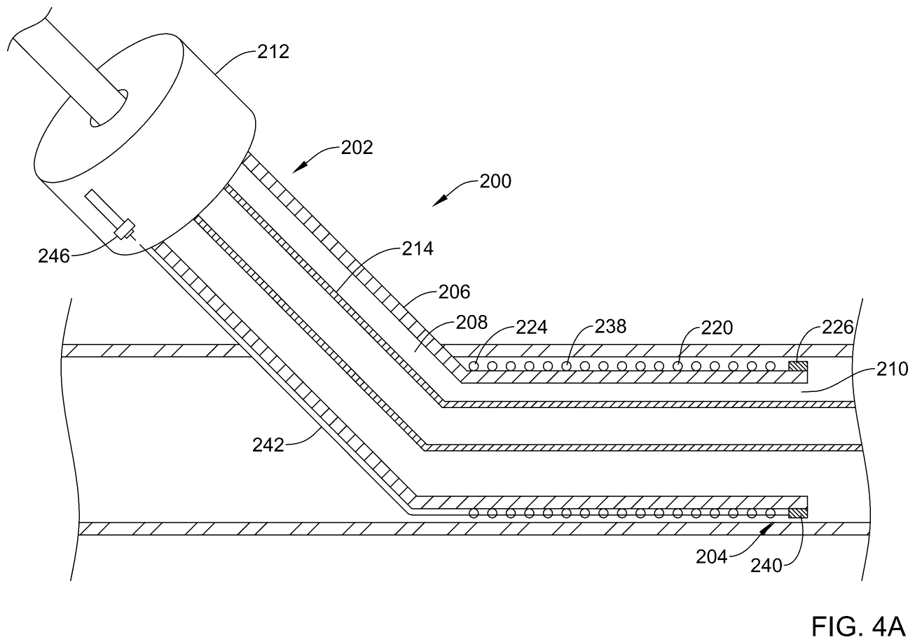

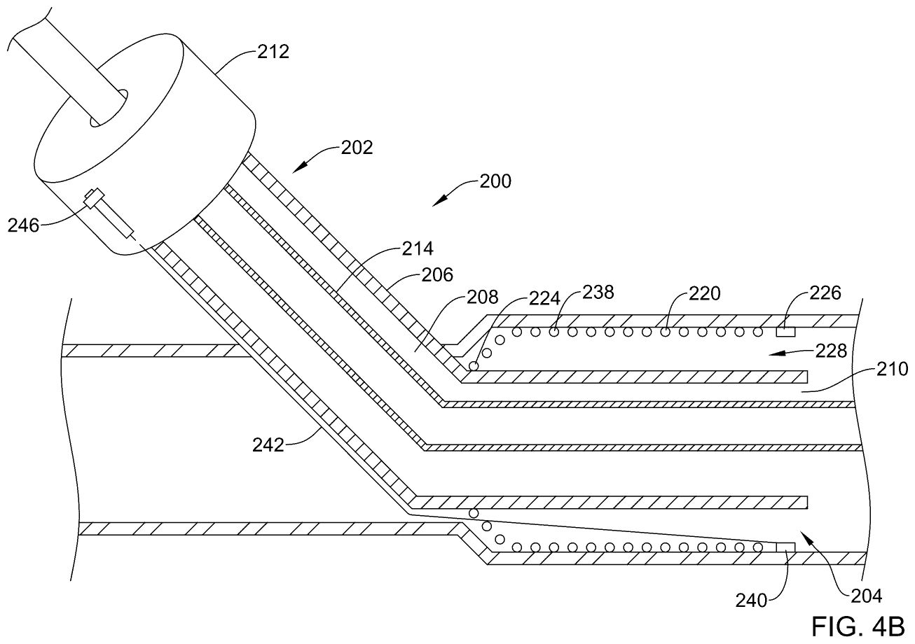

Some large bore access devices may have larger distal capsule sections which require introducer sheaths to have diameters or cross-sectional dimensions large enough to accommodate the cross-sectional dimension of the distal capsule section. However, larger diameter introducer sheaths may occlude or otherwise inhibit the flow of blood through the vessel which may lead to critical limb ischemia. It is contemplated that the introducer sheath 100 may include a blood bypass therein to improve blood flow proximal to the introduction point. FIGS. 3A-3B and 4A-4B depict an illustrative introducer sheath including an expandable frame to improve blood flow proximal to the introduction point. FIG. 3A is a side view of an illustrative introducer sheath 200 having an expandable frame 220 in a first or collapsed configuration and FIG. 3B is a side view of the illustrative introducer sheath 200 having the expandable frame 220 in a second or expanded configuration. FIG. 4A is a partial cross-sectional view of the illustrative introducer sheath 200 extending into a blood vessel V with the expandable frame 220 in the first or collapsed configuration and FIG. 4B is a partial cross-sectional view of the illustrative introducer sheath 200 extending into a blood vessel V with the expandable frame 220 in the second or expanded configuration.

The introducer sheath 200 includes a proximal end region 202 proximate a proximal end of the introducer sheath 200 and a distal end region 204 proximate a distal end of the introducer sheath 200 that is opposite the proximal end region 202. While FIGS. 4A-4B show the distal end region 204 of the introducer sheath 200 proximal to a distal end region (not explicitly shown) of the medical device 214 (FIGS. 4A and 4B), the distal end region 204 of the introducer sheath 200 may extend distal to a distal end region of the medical device 214 or may be adjacent to or near the distal end region of the medical device 214. An elongate shaft 206 of the introducer sheath 200 extends between the proximal end region 202 and the distal end region 204, and the elongate shaft 206 defines a lumen 208 of the introducer sheath 200. The introducer sheath 200 includes a proximal opening (not shown) adjacent the proximal end region 202 and a distal opening 210 adjacent the distal end region 204, with the lumen 208 extending from the proximal opening to the distal opening 210. The introducer sheath 200, or components thereof, may be formed by various materials, such as polymeric and/or metallic materials. In some instances, the introducer sheath 200, such as the elongate shaft of the introducer sheath 200, may include an additional surface coating, such as but not limited to, silicone, polyethylene terephthalate (PET), or other applicable polymer.

A hemostasis valve hub 212 (hereinafter “hub 212” for brevity) may be provided at the proximal end region 202 to provide access to the lumen 208 of the introducer sheath 200. The hub 212 may be configured for hemostasis by, for example, helping to prevent blood from leaking out of the introducer sheath 200 during use. For example, a medical device 214, such as a catheter or blood pump, may be inserted through the hub 212 and lumen 208 of the introducer sheath 200 and into the blood vessel V, and the hub 212 may maintain hemostasis between the medical device 214, the introducer sheath 200, and the external surroundings.

In some embodiments, the elongate shaft 206 of the introducer sheath 200 may include an expandable frame 220 disposed over and extending around an outer surface of the elongate shaft 206 adjacent to the distal end region 204 thereof. Generally, the expandable frame 220 may be movable between a radially collapsed delivery configuration and a radially expanded deployed configuration. In some instances, the expandable frame 220 may be formed from an elongated tubular member 222. While the expandable frame 220 is described as generally tubular, it is contemplated that the expandable frame 220 may take any cross-sectional shape desired. The expandable frame 220 may have a first, or proximal, end 224, a second, or distal, end 226, and an intermediate region disposed between the first end 224 and the second end 226. The expandable frame 220 may include a lumen 228 extending from a first opening adjacent the first end 224 to a second opening adjacent to the second end 226 to allow for the passage of blood or other fluids therethrough.

The expandable frame 220 may be radially expandable from a first radially collapsed configuration (FIGS. 3A and 4A) to a second radially expanded configuration (FIGS. 3B and 4B). The expandable frame 220 may be structured to expand in diameter or cross-sectional dimension to apply a radially outward pressure to increase a diameter or cross-sectional dimension of the vessel V to increase blood flow proximal to the introduction point. For example, in the radially collapsed configuration the expandable frame 220 may have first diameter (or cross-sectional dimension) 230 and in the radially expanded configuration, the expandable frame 220 may have a second diameter (or cross-sectional dimension) 232 greater than the first diameter 230. In some embodiments, the second diameter 232 may be a predetermined diameter. For example, a predetermined second diameter 232 may be in the range of about 4.5 millimeters (mm) to about 6.5 mm. It is contemplated that the expandable frame 220 may be configured to expand to a predetermined diameter greater than an inner diameter of the vessel V in which it is to be positioned. This helps increase blood flow proximal to the introduction point. Thus, a predetermined second diameter 232 may be determined, at least in part, by a desired introduction location and may be less than 4.5 mm or greater than 6.5 mm, as desired. In some examples, the second diameter 232 may be selected to increase the diameter of the vessel V by in the range of about 10% to 15%. However, this is not required. In some cases, the expandable frame 220 may be incrementally expanded to allow the expandable frame 220 to be expanded to a desired second diameter 232. For example, the actuation system (including an actuation member 242 and an actuation member 246) may include one or more intermediate locking points to secure the expandable frame 220 at an intermediate expansion having a diameter between the first diameter 230 and the second diameter 232.

In some embodiments, the expandable frame 220 may retract in longitudinal length as it is expanded. For example, the expandable frame 220 may have a first length 234 in the radially collapsed configuration and a second length 236 shorter than the first length 234 in the radially expanded configuration. However, this is not required. In some embodiments, the first and second lengths 234, 236 may be the same or similar. It is contemplated that the first and/or second lengths 234, 236 may be approximately the same as a length of the elongate shaft 206 that is disposed within the vessel V. However, this is not required. The first and/or second lengths 234, 236 may be longer or shorter than a length of the elongate shaft 206 that is disposed within the vessel V. In some embodiments, the first and/or second lengths 234, 236 may be selected such that a portion of the expandable frame 220 extends exterior to the body. For example, the expandable frame 220 may extend over an entirety of the elongate shaft 206 (e.g., from the proximal end region 202 to the distal end region 204). In some cases, the first and/or second lengths 234, 236 may vary based on the anatomical access location. It is contemplated that the first and/or second lengths 234, 236 of the expandable frame 220 may be in the range of about 11 centimeters (cm) to about 35 cm. However, the expandable frame 220 may be shorter than 11 cm or longer than 35 cm, as desired.

The tubular member 222 of the expandable frame 220 may have a scaffold structure, fabricated from one or more, or a plurality of interwoven filaments or struts 238. The scaffold structure may extend from the first end 224 to the second end 226 of the expandable frame 220. For example, the scaffold structure, and thus the filament(s) thereof, may extend continuously from the first end 224 to the second end 226 of the expandable frame 220. In some embodiments, the expandable frame 220 may be formed with one filament interwoven with itself (e.g., knitted) to form the scaffold structure. In other embodiments, the expandable frame 220 may be formed with several interwoven filaments (e.g., braided) to form the scaffold structure. Thus, in such instances one or more of the filament(s) forming the scaffold structure may extend continuously from the first end 224 to the second end 226 of the expandable frame 220. In still another embodiment, the expandable frame 220 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 224 to the second end 226 of the expandable frame 220.

It is contemplated that the scaffold structure, e.g., the filaments and/or struts, of the expandable frame 220 can be made from a number of different materials such as, but not limited to, metals, metal alloys, shape memory alloys, and/or polymers, as desired, enabling the expandable frame 220 to be expanded into shape when accurately positioned within the body. In some instances, the material may be selected to enable the expandable frame 220 to be removed with relative ease as well. For example, the expandable frame 220 can be formed from alloys such as, but not limited to, nitinol and Elgiloy®. Depending on the material selected for construction, the expandable frame 220 may be self-expanding or require an external force to radially expand the expandable frame 220. In some embodiments, filaments may be used to make the expandable frame 220, which may be composite filaments, for example, having an outer shell made of nitinol and having a platinum core. It is further contemplated that the filaments of the expandable frame 220 may be formed from polymers including, but not limited to, polyethylene terephthalate (PET).

One of the proximal or distal ends 224, 226 of the expandable frame 220 may be fixedly secured to the elongate shaft 206 while the other of the proximal or distal ends 224, 226 may be movably disposed relative to the elongate shaft 206. This may allow the expandable frame 220 to move between the radially collapsed configuration for insertion and removal and the radially expanded configuration while the sheath 200 is in use within the body. In the illustrated embodiment the proximal end 224 of the expandable frame 220 may be fixedly secured to elongate shaft 206 while the distal end 226 is movably disposed relative to the elongate shaft 206. The reverse configuration in which the distal end 226 is fixedly secured to the elongate shaft 206 and the proximal end 224 is movable is also contemplated. It is contemplated that the expandable frame 220 may be coupled to the elongate shaft 206 using a number of different techniques, including, but not limited to, adhesives, hot melting, thermal welding, heat shrinking, welding, soldering, brazing, or the like. In some cases, the movable end (e.g., the distal end 226 in the illustrated embodiment) may include a collar 240 extending around a perimeter thereof. The collar may extend around an entirety of the perimeter or less than an entirety of the perimeter thereof. The collar 240 may be configured to be axially displaced along a longitudinal axis of the elongate shaft 206. It is contemplated that the collar 240 may be formed from the struts 238 or as a single monolithic structure with the tubular member 222. In other examples, the collar 240 may be formed as separate structure and subsequently coupled to or secured to the tubular member 222. It is contemplated that the collar 240 may be secured to the tubular member 222 using a number of different techniques, including, but not limited to, adhesives, hot melting, thermal welding, heat shrinking, welding, soldering, brazing, or the like.

Positioning the collar 240 around the perimeter of the expandable frame 220 may allow the expandable frame 220 to be axially displaced with an actuation member 242. The actuation member 242 may be a pull wire, filament, ribbon, or the like extending from a distal end 244 coupled to the collar 240 to a proximal end (not explicitly shown) coupled to an actuation mechanism 246. The actuation mechanism 246 may be positioned within or adjacent to the hub 212. However, this is not required. The actuation mechanism 246 may be positioned along an exterior of the elongate shaft 206 or within the lumen of the elongate shaft 206, if so desired. Some illustrative actuation mechanisms 246 may include but are not limited to, toggles, thumb switches, roller wheels, or the like. The actuation mechanism 246 may be movable to move the expandable frame 220 between a first radially collapsed configuration and a second radially expanded configuration. In one illustrative example, the actuation mechanism 246 may be distally advanced to distally advance the actuation member 242 and thus the distal collar 240 to position the expandable frame 220 in the elongated radially collapsed configuration. The actuation mechanism 246 may be proximally retracted to proximally retract the actuation member 242 and thus the distal collar 240 to foreshorten the expandable frame 220 and move the expandable frame 220 to the radially expanded configuration. However, in other examples, the actuation mechanism 246 may be proximally retracted to collapse the expandable frame 220 and distally advanced to expand the expandable frame 220. The hub 212 may include visual indicia configured to indicate whether the expandable frame is in a radially expanded or radially collapsed configuration. Visual indicia may include, but is not limited to, colors, shapes, patterns, letters, words, etc.

The actuation member 242 may be configured to extend along an outer surface of the elongate shaft 206. In some cases, the actuation member 242 may extend through the lumen 228 of the expandable frame 220. In other examples, the actuation member 242 may extend along an outer surface of the expandable frame 220. In other examples, the actuation member 242 may be configured to extend within the lumen 208 of the elongate shaft 206 for at least a portion of the length of the actuation member 242. The actuation member 242 may exit the lumen 208 of the elongate shaft 206 through an opening or aperture extending through a side wall of the elongate shaft 206 adjacent to the expandable frame 220 such that the actuation member 242 may be secured to the collar 240.

The expandable frame 220 may be in the radially collapsed configuration for insertion and/or removal of the introducer sheath 200. Once the introducer sheath 200 is in position within the vessel V, the actuation mechanism 246 may be actuated (e.g., proximally retracted in the illustrated embodiment) to radially expand the expandable frame 220. The expandable frame 220 may be fully expanded or partially expanded, as desired. In the expanded configuration, the expandable frame 220 may have a diameter or cross-sectional dimension that is greater than the diameter of the vessel V. Thus, as the expandable frame 220 is expanded, the expandable frame 220 may exert a radially outward biasing force on the wall of the vessel V to expand the diameter of the vessel V. This may increase the amount of blood perfused past the introducer sheath 200 relative to a device free from the expandable frame 220. The expandable frame 220 may be radially collapsed for removal of the introducer sheath 200.

In some embodiments, the expandable frame 220 may include a drug eluting coating or therapeutic coating disposed on one or more surfaces thereof. In some cases, the drug coating or coating composition may include a direct oral anticoagulant (DOAC) on a surface of the expandable frame 220. DOACs bind directly to specific clotting factors. Examples of DOACs include apixaban, rivaroxaban, edoxaban, betrixaban, and argatroban, which directly bind to factor Xa, and dabigatran, which directly binds to factor IIa. However, other beneficial agents may include anti-thrombotic agents, anti-proliferative agents, anti-inflammatory agents, anti-migratory agents, agents affecting extracellular matrix production and organization, antincoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

More specific drugs or therapeutic agents that may be used with the drug eluting coatigs include apixaban, rivaroxaban, edoxaban, dabigatran, betrixaban, argatroban, paclitaxel, rapamycin, sirolimus, everolimus, tacrolimus, heparin, diclofenac, aspirin, Epo D, dexamethasone, estradiol, halofuginone, cilostazol, geldanamycin, ABT-578 (Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others.

In some embodiments, the drug may be a macrolide immunosuppressive (limus) drug. In some embodiments, the macrolide immunosuppressive drug is rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4'S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy) ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), or derivative, isomer, racemate, diastereoisomer, prodrug, hydrate, ester, or analog thereof. Other drugs may include anti-inflammatory agents such as dexamethasone, prednisolone, corticosterone, budesonide, estrogen, sulfasalazine, mesalamine, and analogues thereof; antineoplastic/antiproliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof; anti-coagulants; and growth factors.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A hub and sheath assembly, comprising:
a valve hub including a lumen extending therethrough;
an elongate shaft extending from a proximal end region to a distal end region and including a lumen extending therebetween, the elongate shaft coupled to the valve hub; and
an expandable frame extending around an outer surface of the elongate shaft, the expandable frame extending from a proximal end to a distal end;
wherein the expandable frame is configured to move between a radially collapsed configuration and a radially expanded configuration.

2. The hub and sheath assembly of claim 1, wherein the proximal end of the expandable frame is fixedly coupled to the elongate shaft.

3. The hub and sheath assembly of claim 1, wherein the distal end of the expandable frame is movably disposed relative to the elongate shaft.

4. The hub and sheath assembly of claim 1, wherein the distal end of the expandable frame is fixedly coupled to the elongate shaft.

5. The hub and sheath assembly of claim 1, wherein the proximal end of the expandable frame is movably disposed relative to the elongate shaft.

6. The hub and sheath assembly of claim 1, further comprising a collar extending around a circumference of the expandable frame.

7. The hub and sheath assembly of claim 6, wherein the collar is adjacent to the distal end of the expandable frame.

8. The hub and sheath assembly of claim 6, wherein the collar is adjacent to the proximal end of the expandable frame.

9. The hub and sheath assembly of claim 1, further comprising an actuation member extending from a distal end secured to the expandable frame to a proximal end.

10. The hub and sheath assembly of claim 9, further comprising an actuation mechanism coupled to the proximal end of the actuation member.

11. The hub and sheath assembly of claim 10, wherein the actuation mechanism is configured to axially displace to the actuation member to move the expandable frame between the radially collapsed configuration and the radially expanded configuration.

12. The hub and sheath assembly of claim 10, wherein the actuation mechanism is disposed within the valve hub.

13. The hub and sheath assembly of claim 9, wherein the actuation member extends along an outer surface of the elongate shaft.

14. The hub and sheath assembly of claim 9, wherein the actuation member is coupled to the collar.

15. The hub and sheath assembly of claim 1, wherein the expandable frame comprises two or more interwoven filaments.

16. A hub and sheath assembly, comprising:
a valve hub including a lumen extending therethrough;
an elongate shaft extending from a proximal end region to a distal end region and including a lumen extending therebetween, the elongate shaft coupled to the valve hub; and
an expandable frame extending around an outer surface of the elongate shaft, the expandable frame extending from a proximal end fixedly secured to the elongate shaft to a distal end movably disposed relative to the elongate shaft;
wherein the expandable frame is configured to be actuated between a radially collapsed configuration and a radially expanded configuration.

17. The hub and sheath assembly of claim 16, further comprising an actuation member extending from a distal end secured to the distal end of the expandable frame to a proximal end.

18. The hub and sheath assembly of claim 17, further comprising an actuation mechanism coupled to the proximal end of the actuation member.

19. The hub and sheath assembly of claim 18, wherein the actuation mechanism is configured to axially displace to the actuation member to move the expandable frame between the radially collapsed configuration and the radially expanded configuration.

20. A hub and sheath assembly, comprising:
a valve hub including a lumen extending therethrough;
an elongate shaft extending from a proximal end region to a distal end region and including a lumen extending therebetween, the elongate shaft coupled to the valve hub;
an expandable frame extending around an outer surface of the elongate shaft, the expandable frame extending from a proximal end fixedly secured to the elongate shaft to a distal end movably disposed relative to the elongate shaft;
a collar disposed around the distal end of the expandable frame;
an actuation mechanism disposed within the valve hub; and
an actuation member extending from a distal end coupled to the collar to a proximal end coupled to the actuation mechanism;
wherein the actuation mechanism is actuatable to move the expandable frame between a radially collapsed configuration and a radially expanded configuration.
